(19) United States
(12) Patent Application Publication
(10) Pub. No.: US 2004/0122337 A1
(43) Pub. Date: Jun. 24, 2004

(54) Device for temperatue and/or other human and animal physiological functions control

(76) Inventor: Antonino Nostro, Segrate (IT)

Correspondence Address: Striker, Striker & Stenby, 103 East Neck Road, Huntington, NY, 11743, US

(21) Appl. No.: 10/474,091

(22) PCT Filed: Apr. 10, 2002
(86) PCT No.: PCT/IT02/00228

(30) Foreign Application Priority Data
Apr. 12, 2001 (IT) CZ2001A000005

(51) Int. Cl. A61B 5/00

(52) U.S. Cl. 600/549

(57) ABSTRACT

Device for temperature and/or other human and animal physiological functions control consisting in a sensor, regulators of minimum and maximum temperature and for other physiological functions, ringer alarm that goes off when an established level is exceeded, an electronic circuit, battery supplying necessary energy to the device. The device may be equipped with an acoustic alarm, a display and an audio system, which states the achieved value of the physiological functions.

Subject of this patent is a device for temperature and/or other human and animal physiological functions control, such as heart rate, systolic and diastolic blood pressures, etc.

The device is made up of: a sensor, minimum and maximum body temperature and others physiological function level regulators, an alarm activated when an established threshold is exceeded, an electronic circuit, a battery supplying the necessary energy for the device to work.

The device may be equipped with an acoustic alarm, a display and an audio system, which states the achieved value of the physiological functions.

Control devices on the market today are for the most part unwieldy and therefore cannot be carried or worn effortlessly while the smaller ones have a restricted performance.

International patent application nr. WO9009570 is a device that monitors body temperature. It is composed of a sensor that is to be applied to the body of a child or adult. The sensor is connected by a wire to the control unit, separated from the sensor that is equipped with leds, temperature regulator elements and sound alarms. When the temperature exceeds a certain peak or falls below an established minimum the leds begin to blink and the sound alarm goes off.

One of the first drawbacks of the this device, described in patent WO9009570 is the fact that the sensor is detached from the control unit and that the two parts (sensor+control unit) are connected through a wire which may be dangerous for the child while he/she is asleep or at best may disturb the person wearing the device when lying down or standing.

Furthermore, the device described in WO9009570 does not allow to know, which is the body temperature, but it merely allows knowing if the peak temperature has been exceeded or if the temperature has dropped beneath the minimum.

Finally, said control device does not allow monitoring other human or animal physiological functions such as arterial blood pressure, heart rate etc.

Main purpose of this invention is to create a device for temperature and/or other human and animal physiological functions control, made of a sensor, regulators for peak and minimum temperatures and/or for other physiological animal and human functions, an alarm activated when an established threshold is exceeded; an electronic circuit; a battery supplying necessary working energy, with the sensor, the regulators, the alarm and the electronic circuit and the battery that are contained in an adhesive plaster that is to be applied on the human or animal body, showing, through the said device, the temperature and/or other controlled human physiological functions.

Another feature is that the value of the temperature and/or the other human physiological functions controlled are measured by varying the value of the regulator and reading the value reached when the alarm stops ringing.

Another characteristic is that it is provided with a display that allows immediate level reading of the temperature and/or the other human physiological functions.

Another feature is that said control device is endowed with an audio system that with the push of a button vocally gives the registered level of the temperature and/or other physiological functions.

Another characteristic is that said device may be powered by electrical current transformed into low tension and connected to the sensor.

Another feature is that a wire carries out the connection;

Another feature is that data is transmitted wirelessly through a transreceiver system.

Another feature is that the measured level is marked at a preset time and registered.

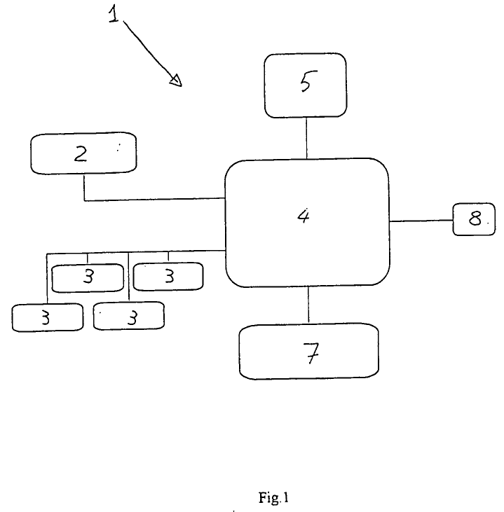

Further characteristics and advantages of the invention will be more evident through the description of the preferred but not exclusive realisation methods, of the device itself, illustrated, just as an indication, but not as a limitation with the aid of the enclosed drawings: FIGS. 1-2-3-4-5:

FIG. 1 shows a working scheme of the device subject of the invention.

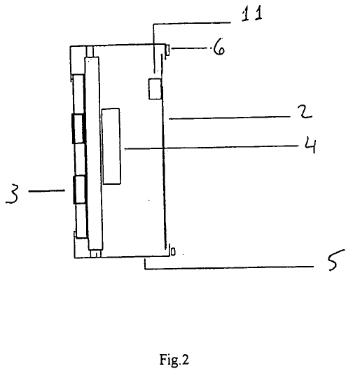

FIG. 2 shows a section of the device subject of the invention.

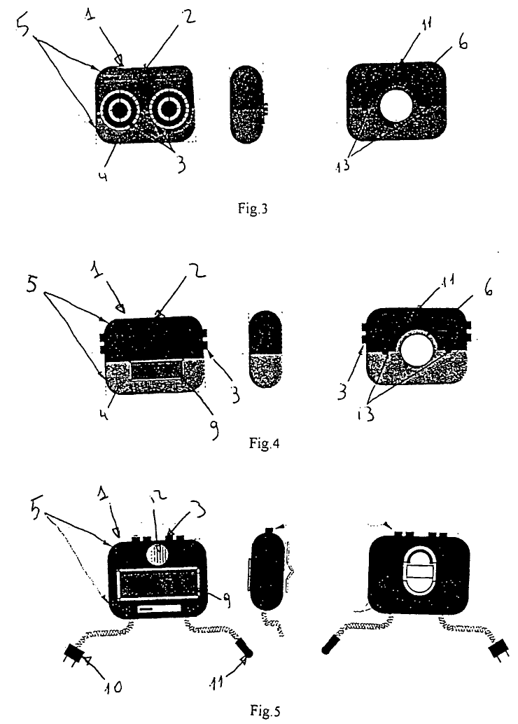

FIG. 3 shows three views of the device, a front view, a rear view, and a lateral view of a realization of the device subject the invention.

FIG. 4 shows three views of the device, a front view, a rear view, and a lateral view of the device subject of the invention with a display.

FIG. 5 shows three views of the device, a front view, a rear view, and a lateral view of the device subject of the invention with an electrical grid.

Device 1 (see FIG. 3) containing an integrated circuit (4) inserted in a shelter box (5) suitable in particular for children watches over night and day body temperature putting into motion an acoustic signal (2) when the established minimum and maximum values are exceeded, that can be programmed through two regulators (3).

These regulators (3) allow taking the actual body temperature that could be also above the established level, this reading may be carried out also without a display, to obtain temperature reading it is sufficient to wait a few instants after the device begins to ring and, after moving the temperature regulator control (3) the moment in which it stops ringing on the regulator one can read the corresponding temperature, that is reached by the body.

The device, therefore is not only a temperature alarm but also a temperature reader as well, with a new made system making it shock resistant and non-breakable. It can be built on a smaller scale and wired in a plain round plaster (6). It can be applied both to constantly keep watch on people and animals.

The functioning is as follows (see FIG. 1)

It is sufficient to regulate the minimum and maximum temperatures through regulators (3) to the required values, the minimum temperature can serve as an alarm in case of sudden infant death syndrome or body freezing, the maximum temperature to put in alarm in case of high fever.

Device (1) is applied through an adhesive (6) as if it were a plaster, in convenient positions of the body, also provided with airing transpiration holes (13), the thermal sensor (7) therefore controls the temperature every 10/15 seconds, making the alarm ring when the established level is exceeded by ringer (2) and nonetheless continuing its control of the temperature, therefore to stop the alarm it is necessary to move the regulator (3) to disconnect the ringer, into the disconnection point corresponding to the actual temperature.

Battery life (8) is about 3 years long related to its very low consumption. It may also be equipped with a display (9) in order to have a visual reading of the temperature (see FIGS. 4 and 5).

Ringer (2) could be piezoelectric, therefore also becoming the metallic measuring base upon the back of which is applied the temperature sensor, or else a metallic base can be used in its place and a normal ringer, similar to the cellular telephone ringer, can be placed on the circuit, in order to have a much stronger sound.

To reduce size, the entire circuit and all components can be, upon surface treatment, cast and printed on the backside of the piezoelectric alarm base or of the metallic base.

The device (FIG. 5) may be equipped with an external electrical plug (10) and an independent sensor (11) either wired or connected through a transreceiver system.

Furthermore, it has the possibility of minimum and maximum temperature registration (memories) in 24 hour time periods and regular programmed memories during the day.

A vocal speaker (12) can be added in order to have, by pressing a key the temperature repeated orally; this feature is especially advantageous for the visually impaired.

The precision level that is obtainable by this device in keeping with the invention is very high.

This invention allows several advantages and overcoming obstacles that could not have been defeated with the currently sold systems.

1) Device for temperature and/or other human and animal physiological functions control consisting in a sensor, ringer alarm that goes off when an established level is exceeded, an electronic circuit, a battery supply necessary energy to the device characterized by the fact that, in combination, it has regulators of minimum and maximum temperature and of other physiological functions; that the sensor, the alarm, the electronic circuit, the battery, and the regulators are contained all together within an adhesive plaster that is to be applied on human or animal body and that it has an audio system that by pressing a key repeats orally the temperature or other physiological functions when the minimum or maximum temperature exceeded.

2) Device for temperature and/or other human and animal physiological functions control according to claim 1) characterized by the fact that checked levels are controlled regularly and recorded in the memory of the device.

3) Device for temperature and/or other human and animal physiological functions control according to claim 1) characterized by the fact that temperature range or other physiological functions are measured by tuning the regulators and reading the level marked when the ringer stops.

4) Device for temperature and/or other human and animal physiological functions control according to claim 1) characterized by the fact that there is a display for instant reading of temperature and other physiological functions under control.

5) Device for temperature and/or other human and animal physiological functions control according to claim 1) characterized by the fact that the termal sensor controls the temperature every 10/15 seconds.
